SEQUENCE LISTING (26 sequences)

SEQ ID NO: 1 - protein, 8 aa, Artificial Sequence
  Peptide consensus sequence
XXXXXXXX

SEQ ID NO: 2 - protein, 8 aa, Artificial Sequence
  Peptide consensus sequence
XXXXXXXX

SEQ ID NO: 3 - protein, 7 aa, Artificial Sequence
  Prion protein fragment
KKRPKPG

SEQ ID NO: 4 - protein, 11 aa, Artificial Sequence
  Peptide consensus sequence
XXRXXXXXXXX

SEQ ID NO: 5 - protein, 5 aa, Artificial Sequence
  Peptide consensus sequence
KXRXX

SEQ ID NO: 6 - protein, 9 aa, Artificial Sequence
  Synthesized cationic peptide sequence
RKKRRQRRR

SEQ ID NO: 7 - protein, 86 aa, Human immunodeficiency virus type 1
MEPVDPRLEPWKHPGSQPKTACTNCYCKKCCFHCQVCFITKALGISYGRKKRRQRRRPPQGSQTHQVSLSKQPTSQSRGDPTGPKE

SEQ ID NO: 8 - protein, 5 aa, Artificial Sequence
  Peptide Linker Sequence
GGGGS

SEQ ID NO: 9 - protein, 5 aa, Artificial Sequence
  Peptide Linker Sequence
GGGGS

SEQ ID NO: 10 - protein, 12 aa, Artificial Sequence
  Peptide Linker Sequence
GKSSGSGSESKS

SEQ ID NO: 11 - protein, 14 aa, Artificial Sequence
  Peptide Linker Sequence
GSTSGSGKSSEGKG

SEQ ID NO: 12 - protein, 18 aa, Artificial Sequence
  Peptide Linker Sequence
GSTSGSGKSSEGSGSTKG

SEQ ID NO: 13 - protein, 18 aa, Artificial Sequence
  Peptide Linker Sequence
GSTSGSGKPGSGEGSTKG

SEQ ID NO: 14 - protein, 14 aa, Artificial Sequence
  Peptide Linker Sequence
EGKSSGSGSESKEF

SEQ ID NO: 15 - protein, 20 aa, Artificial Sequence
  HA2 analog
GLFGAIAGFIEGGWTGMIDG

SEQ ID NO: 16 - protein, 20 aa, Artificial Sequence
  HA2 analog
GLFEAIAEFIEGGWEGLIEG

SEQ ID NO: 17 - protein, 10 aa, Artificial Sequence
  Fragment DV3 peptide ligand
LGASWHRPDK

SEQ ID NO: 18 - protein, 42 aa, Artificial Sequence
  Fusion Polypeptide
LGASWHRPDKGRRRQRRKRGKKHRSTSQGKKSKLHSSHARSG

SEQ ID NO: 19 - protein, 42 aa, Artificial Sequence
  Fusion Polypeptide
LGASWHRPDKGRRRQRRKRGKKHRSTSQGEASELHSSHARSG

SEQ ID NO: 20 - protein, 11 aa, Artificial Sequence
  Fragment DV3 peptide ligand
LGASWHRPDKG

SEQ ID NO: 21 - protein, 32 aa, Artificial Sequence
  Fusion Polypeptide
RRRQRRKKRGKKHRSTSQGKKSKLHSSHARSG

SEQ ID NO: 22 - protein, 21 aa, Artificial Sequence
  Fusion Polypeptide
LGASWHRPDKGRRRQRRKKRG

SEQ ID NO: 23 - protein, 22 aa, Artificial Sequence
  Fusion Polypeptide Fragment
KKHRSTSQGKKSKLHSSHARSG

SEQ ID NO: 24 - protein, 33 aa, Artificial Sequence
  Fusion Polypeptide
LGASWHRPDKGKKHRSTSQGKKSKLHSSHARSG

SEQ ID NO: 25 - protein, 18 aa, Artificial Sequence
  Fusion Polypeptide
RKKRRQRRRGPVKRRLFG

SEQ ID NO: 26 - protein, 29 aa, Artificial Sequence
  Fusion Polypeptide
LGASWHRPDKGRKKRRQRRRGPVKRRLFG

(19) United States
(12) Patent Application Publication
(10) Pub. No.: US 2009/0098049 A1
(43) Pub. Date: Apr. 16, 2009

(54) Targeting transducible molecules to specific cell types

(75) Inventors: Steven F Dowdy, La Jolla, CA (US); Eric L. Snyder, Brookline, MA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: The Regents of the University of California, San Diego, CA (US)

(21) Appl. No.: 11/662,170

(22) PCT Filed: Sep. 7, 2005
(86) PCT No.: PCT/US05/31539

§ 371 Date: Oct. 4, 2007

Related U.S. Application Data
(60) Provisional application No. 60/607,882, filed on Sep. 7, 2004.

(51) Int. Cl. A61K 49/00; C07K 7/00; C12N 9/72; C12N 9/70; C12N 15/00; C12N 15/11; C12N 5/06; A61K 38/02

(52) U.S. Cl. 424/9.1; 530/330; 530/329; 530/328; 530/327; 435/215; 435/216; 435/375; 514/2; 536/23.4; 435/320.1

(57) ABSTRACT

The disclosure provides fusion polypeptides and constructs useful in targeting molecules including diagnostics and therapeutics to a cell type of interest. The fusion constructs include a protein transduction domain, a ligand domain and a cargo domain. Also provided are methods of treating disease and disorders such as cell proliferative disorders.

### CROSS REFERENCE TO RELATED APPLICATIONS

The application claims priority under 35 U.S.C. §119 to U.S. Provisional Application Ser. No. 60/607,882, filed Sep. 7, 2004, the disclosure of which is incorporated herein by reference.

### STATEMENT REGARDING FEDERALLY SPONSORED RESEARCH

This invention was funded in part by Grant No. CA96098 awarded by National Institutes of Health. The government may have certain rights in the invention.

### TECHNICAL FIELD

This disclosure relates to fusion polypeptides comprising a transduction moiety and a therapeutic or diagnostic moiety.

### BACKGROUND

Eukaryotic cells contain several thousand proteins, which have been, during the course of evolution, selected to play specific roles in the maintenance of virtually all cellular functions. Not surprisingly then, the viability of every cell, as well as the organism on the whole, is intimately dependent on the correct expression of these proteins. Factors which affect a particular protein's function, either by mutations or deletions in the amino acid sequence, or through changes in expression to cause over-expression or suppression of protein levels, invariably lead to alterations in normal cellular function. Such alterations often directly underlie a wide variety of genetic and acquired disorders. Consequently, the ability to target and selectively inhibit or kill cells comprising mutations that result in cell proliferative disorders would help to control such diseases and disorders.

In practice however, the direct intracellular delivery of these agents has been difficult. This is due primarily to the bioavailability barrier of the plasma membrane, which effectively prevents the uptake of the majority of peptides and proteins and other agents by limiting their passive entry.

Traditionally, approaches to modulate protein function have largely relied on the serendipitous discovery of specific drugs and small molecules which could be delivered easily into the cell. However, the usefulness of these pharmacological agents is limited by their tissue distribution and unlike “information-rich” proteins, they often suffer from poor target specificity, unwanted side-effects, and toxicity. Likewise, the development of molecular techniques for gene delivery and expression of proteins has provided for advances in our understanding of cellular processes but has been of little benefit for the management of genetic disorders (Robbins et al., Trends Biotechnol. 16:35-40, 1998; Robbins and Ghivizzani, Pharmacol. Ther. 80:35-47, 1998).

### SUMMARY

The invention provides a fusion polypeptide comprising: (a) a protein transduction domain (PTD), the transduction domain comprising a membrane transport function; (b) a ligand domain comprising a ligand specific for an extracellular protein (e.g., a receptor); and (c) a heterologous domain (e.g., a therapeutic and/or diagnostic agent), wherein the PTD is operably linked to the ligand domain and the heterologous domain.

The invention also provides a method of introducing a therapeutic and/or diagnostic agent into a target cell, the method comprising contacting the cell with a fusion polypeptide comprising: (a) a protein transduction domain (PTD), the transduction domain comprising a membrane transport function; (b) a ligand domain comprising a ligand specific for an extracellular receptor; and (c) a therapeutic and/or diagnostic agent, wherein the PTD is operably linked to the ligand domain and the therapeutic and/or diagnostic agent.

The disclosure provides compositions and methods for treating cell proliferative disorders overexpressing a receptor related to the cell proliferative disorder and transducing such cells with a fusion polypeptide comprising a transducible peptide moiety, a ligand for a receptor and an anti-proliferative or diagnostic agent.

The invention provides a fusion polypeptide comprising (a) a protein transduction domain (PTD), the transduction domain comprising a membrane transport function; (b) a ligand domain comprising a ligand specific for an extracellular polypeptide on a cell of interest; and (c) a heterologous domain, wherein the PTD is operably linked to the ligand domain and the heterologous domain. In one aspect, the protein transduction domain is selected from the group consisting of a polypeptide comprising a herpesviral VP22 domain; a polypeptide comprising a human immunodeficiency virus (HIV) TAT domain; a polypeptide comprising a homeodomain of an Antennapedia protein (Antp HD) domain; an N-terminal cationic prion protein domain; and functional fragments thereof. For example, the protein transduction domain comprises a sequence selected from the group consisting of SEQ ID NO:7 from amino acid 47-57; B1-X₁-X₂-X₃-B₂-X₄-X₅-B₃, wherein B₁, B₂, and B₃ are each independently a basic amino acid, the same or different and X₁, X₂, X₃, X₄ and X₅ are each independently an alpha-helix enhancing amino acid the same or different (SEQ ID NO:1); B₁-X₁-X₂-B₂-B₃-X₃-X₄-B₄, wherein B₁, B₂, B₃, and B₄ are each independently a basic amino acid, the same or different and X₁, X₂, X₃, and X₄ are each independently an alpha-helix enhancing amino acid the same or different (SEQ ID NO:2); X-X-R-X-(P/X)-(B/X)-B-(P/X)-X-B-(B/X), wherein X is any alpha helical promoting residue such as alanine; P/X is either proline or X as previously defined, B is a basic amino acid residue and B/X is either B or X as defined above (SEQ ID NO:4); a sequence of about 7 to 10 amino acids and containing KX₁RX₂X₁, wherein X₁ is R or K and X₂ is any amino acid (SEQ ID NO:5); RKKRRQRRR (SEQ ID NO:6); and KKRPKPG (SEQ ID NO:3). In another aspect, the heterologous domain comprises a diagnostic and/or therapeutic agent.

The invention also provides a pharmaceutical composition comprising the fusion polypeptide of the invention.

The invention also provides a method of introducing a therapeutic and/or diagnostic agent in to a target cell, the method comprising contacting the cell with the fusion polypeptide of the invention.

The invention provides a method of treating a cell proliferative disorder in a subject, comprising contacting the subject with a fusion polypeptide of the invention, wherein the heterologous domain comprises an anticellular agent.

The invention further provides a method of identifying a cell comprising a phenotype of interest in a subject, the method comprising contacting the subject with a fusion polypeptide of the invention, wherein the heterologous domain comprises a diagnostic agent.

The invention provides an isolated polynucleotide encoding a fusion polypeptide of the invention, as well as vectors and recombinant host cells comprising the polynucleotide.

The details of one or more embodiments are set forth in the accompanying drawings and the description below. Other features, objects, and advantages will be apparent from the description and drawings, and from the claims.

### BRIEF DESCRIPTION OF THE FIGURES

FIG. 1A-B show that the CXCR4 receptor binding DV3 peptide domain increases the affinity of TAT peptides for CXCR4-expressing lymphoma cells. (a) Sequences of peptides used in some embodiments. All peptides were synthesized using D-isomer residues, except for the TAT-RxL peptides. (b) Human Namalwa lymphoma cells that overexpress CXCR4 receptor were incubated with increasing concentrations of peptide, followed by fluorescent PE-conjugated anti-CXCR4 monoclonal antibody incubation, then analyzed for antibody binding to CXCR4 receptor (mean fluorescence) by flow cytometry. Graph plots relative fluorescence of cells with respect to cells treated with antibody only. Data represents the mean and standard error from three independent experiments.

FIG. 2A-E depict data demonstrating that targeted DV3-TATp53C′ and DV3-TAT-RxL peptides kill CXCR4-expressing lymphoma cells with increased efficacy. (a) Namalwa lymphoma cells were treated with 40 μM TATp53C′ or DV3-TATp53C′ peptide for 48 h. Cell viability was assessed by trypan blue exclusion. Data represents the mean and standard error from three independent experiments. (b) DV3-TATp53C′ and TATp53C′ peptides induce similar level of p53-dependent G₁ cell cycle arrest in CXCR4 non-expressing TA3/St mammary carcinoma cells. Cells were treated with 5 μM peptide for 24 hours and analyzed for DNA content by flow cytometry. (c) DV3-TATp53C′, TATp53C′ and control DV3 peptide (30 μM) have no effect on CXCR4 non-expressing, p53-deficient H1299 lung adenocarcinoma cells. (d) DV3-TAT-RxL is more potent than TAT-RxL in killing CXCR4-expressing Namalwa lymphoma cells. Cells were treated with indicated concentrations of cdk2 antagonists TAT-RxL or DV3-TAT-RxL peptides for 48 h. Cell viability was assessed by trypan blue exclusion. Data represents the mean and standard error from three independent experiments. (e) Namalwa lymphoma (CXCR4+) cells were treated with DV3-TATp53C′, TATp53C′ or DV3-TATp53MUT peptides for 24 hours. Apoptosis was determined by <2N DNA content as measured by flow cytometry and DNA staining.

FIG. 3A-B show DV3 domain enhanced effect requires covalent linkage to TATp53C′ peptide. (a) Addition of DV3-TATp53C′ constituent domains in trans does not recapitulate the effect of DV3-TATp53C′ peptide in cis on lymphoma cells, as indicated. Namalwa lymphoma cells were treated with 30 μM peptide for 48 h. Cell viability was assessed by trypan blue exclusion. Data represents the mean and standard error from three independent experiments. (b) Blockade of CXCR4 receptors by excess DV3 peptide reduces the ability of DV3-TATp53C′ to kill Namalwa lymphoma cells to TATp53C′ level. Cells were treated with 30 μM DV3-TATp53C′ or parental TATp53C′ peptide for 48 h in the presence or absence of a 200 μM excess of DV3 peptide. Cell viability was assessed by trypan blue exclusion. Data represents the mean and standard error from three independent experiments.

FIG. 4A-C show that the enhanced effect by DV3-TATp53C′ targeted peptide requires CXCR4 receptor expression. (a) Flow cytometry analysis of control, CXCR4 non-expressing 293T cells and CXCR4 transfected 293T cells incubated with PE-labeled anti-CXCR4 antibody. (b) and (c) show ectopic expression of CXCR4 in 293T cells enhances efficacy of DV3-TAT-RxL peptide induced cell death. 293T cells were transiently transfected with CXCR4 expression plasmid for 18 hours, followed by peptide treatment for 24 hours. Cell viability was assessed by trypan blue exclusion (b). Apoptosis was measured by DAPI staining for nuclear condensation (c). Data represents the mean and standard error from two independent experiments.

FIG. 5. Targeted DV3-TATp53C′ peptide has enhanced ability to treat mouse model of aggressive, metastatic peritoneal lymphoma. Namalwa lymphoma cells were intraperitoneally injected into SCID mice and allowed to proliferate for 48 hours. Mice were then injected once a day for 12 days with vehicle control (n=10), or 180 nmol DV3-TATp53C′ peptide (n=11), non-targeted, parental TATp53C′ peptide (n=5), or control DV3-only peptide (n=5). Vehicle, non-targeted, parental TATp53C′ peptide and DV3-only peptide treated mice had a median survival of 28, 30 and 27 days, respectively, whereas CXCR4 targeted DV3-TATp53C′ peptide treated mice had a significant increased survival p<0.001) with a median survival of 41 days and 18% long-term survivors (>120 days).

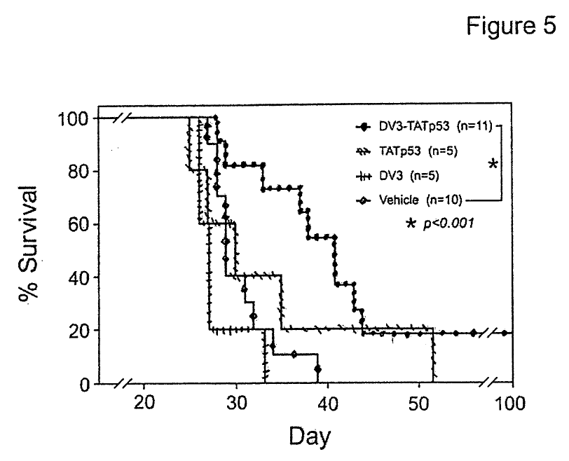

### DETAILED DESCRIPTION

The disclosure provides chimeric/fusion polypeptides comprising a PTD, a ligand, and a heterologous molecule. In one aspect, the chimeric/fusion polypeptide comprises a PTD linked to a ligand (e.g., a receptor ligand), and a heterologous molecule such as a polynucleotide, a small molecule, or a heterologous polypeptide domain. In another aspect, the chimeric/fusion polypeptide comprises a PTD linked to a receptor ligand, and a fusogenic domain.

As used herein and in the appended claims, the singular forms “a,” “and,” and “the” include plural referents unless the context clearly dictates otherwise. Thus, for example, reference to “a target cell” includes a plurality of such cells and reference to “the expression vector” includes reference to one or more transformation vectors and equivalents thereof known to those skilled in the art, and so forth.

Unless defined otherwise, all technical and scientific terms used herein have the same meaning as commonly understood to one of ordinary skill in the art to which this disclosure belongs. Although any methods, cells and genes similar or equivalent to those described herein can be used in the practice or testing of the disclosed methods and compositions, the exemplary methods, devices and materials are now described.

The publications discussed above and throughout the text are provided solely for their disclosure prior to the filing date of the present application. Nothing herein is to be construed as an admission that the inventors are not entitled to antedate such disclosure by virtue of prior disclosure.

An advantage of protein transduction is the intracellular delivery of proteins or agents which are otherwise difficult to transfect and where microinjection is not a possible option. For instance, primary lymphocytes are very difficult to transfect, requiring electroporation of DNA constructs. This process is very inefficient, killing 90-99% of the cells, and yielding protein expression in less than 10% of those which survive.

The ability to deliver functional agents to cells is problematical due to the bioavailability restriction imposed by the cell membrane. That is, the plasma membrane of the cell forms an effective barrier, which restricts the intracellular uptake of molecules to those which are sufficiently non-polar and smaller than approximately 500 daltons in size. Previous efforts to enhance the internalization of proteins have focused on fusing proteins with receptor ligands (Ng et al., Proc. Natl. Acad. Sci. USA, 99:10706-11, 2002) or by packaging them into caged liposomal carriers (Abu-Amer et al., J. Biol. Chem. 276:30499-503, 2001). However, these techniques often result in poor cellular uptake and intracellular sequestration into the endocytic pathway.

The disclosure provides fusion polypeptides and compositions useful in cellular transduction and cellular modulation. The fusion polypeptides of the disclosure comprise a transduction moiety domain comprising a membrane transport function, a targeting ligand and a heterologous domain (e.g., a therapeutic or diagnostic agent).

A number of protein transduction domains/peptides are known in the art and have been demonstrated to facilitate uptake of heterologous molecules linked to the domain (e.g., cargo molecules). Such transduction domains facilitate uptake through a process referred to a macropinocytosis. However, macropinocytosis is a nonselective form of endocytosis that all cells perform. Consequently, this non-selective aspect of protein transduction also results in the majority of the PTD-cargo being transduced into non-target cells in vivo and thereby requires vastly more material. Therefore, pharmacologically speaking, PTDs resemble currently used small molecule therapeutics in their lack of specific delivery to the cells and tissues for which they are intended in vivo.

Similar to chemotherapy, it is likely that most tissues receive only a small fraction of the total non-targeted, TAT-molecule administered. Therefore, even a small increase in the total amount of peptide delivered to target tumor cells could lead to a substantial increase in potency, a decrease in the minimally effective dose and/or a decrease in potential side-effects. Taken together, these observations demonstrate that a multi-domain approach can be used to selectively target fusion polypeptides comprising a PTD domain to a desired cell type. In one aspect, the multi-domain approach of the invention can be used to modulate transducible anticancer peptides to selectively target and kill tumor cells based on receptor overexpression, common to many malignancies. Due to the inherent absence of a size limitation on transduction domains to deliver therapeutic cargo into cells, the invention can be applied reiteratively to refine both the tumor selectivity and killing abilities of multi-domain transducible macromolecules to further enhance therapeutic efficacy.

Tumor cells and other cells having cell proliferative disorders overexpress a variety of receptors on their cell surface, including HER2 receptor in breast cancer, GnRH receptor in ovarian carcinomas and CXCR4 receptor in multiple tumor types. Due to genetic alterations in protein degradation pathways and hypoxic regions of tumors, the CXCR4 chemokine receptor is overexpressed in over 20 different types of tumors, including breast cancer, ovarian cancer, glioma, pancreatic cancer, prostate cancer, AML, B-chronic lymphocytic leukemia, melanoma, cervical cancer, colon carcinoma, rhabdomyosarcoma, astrocytoma, small-cell lung carcinoma, CLL, renal cancer and non-Hodgkin's lymphoma. Therefore, therapeutics that target CXCR4 overexpressing tumor cells may be applicable to malignancies at the earliest stages of oncogenesis.

The invention provides a multi-domain approach to enhance tumor targeting of non-selective PTD-mediated protein transduction delivery. The invention demonstrates that the addition of a ligand targeting domain (e.g., CXCR4 targeting domain (DV3)) enhanced cell specific targeting and in the case of targeted cell killing increases cell killing in, for example, lymphoma cells in a cargo independent fashion, but had no enhanced effect on cells not expressing the target ligands cognate. The increased potency was dependent on cis linkage of a targeting ligand domain to a PTD and heterologous domain. Furthermore, the enhanced cell killing demonstrated in the Examples below demonstrates the applicability of the invention to the targeted delivery of PTD-cargo molecules and broad implications for treating malignant disease by PTD-mediated protein transduction.

The recent discovery of several proteins which could efficiently pass through the plasma membrane of eukaryotic cells has led to the identification of a novel class of proteins from which peptide transduction domains have been derived. The best characterized of these proteins are the Drosophila homeoprotein antennapedia transcription protein (AntHD) (Joliot et al., New Biol. 3:1121-34, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP22 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), and more recently the cationic N-terminal domain of prion proteins. Not only can these proteins pass through the plasma membrane but the attachment of other proteins, such as the enzyme β-galactosidase, was sufficient to stimulate the cellular uptake of these complexes. Such chimeric proteins are present in a biologically active form within the cytoplasm and nucleus. Characterization of this process has shown that the uptake of these fusion polypeptides is rapid, often occurring within minutes, in a receptor independent fashion. Moreover, the transduction of these proteins does not appear to be affected by cell type and can efficiently transduce 100% of cells in culture with no apparent toxicity (Nagahara et al., Nat. Med. 4:1449-52, 1998). In addition to full-length proteins, protein transduction domains have also been used successfully to induce the intracellular uptake of DNA (Abu-Amer, supra), antisense oligonucleotides (Astriab-Fisher et al., Pharm. Res, 19:744-54, 2002), small molecules (Polyakov et al., Bioconjug. Chem. 11:762-71, 2000) and even inorganic 40 nanometer iron particles (Dodd et al., J. Immunol. Methods 256:89-105, 2001; Wunderbaldinger et al., Bioconjug. Chem. 13:264-8, 2002; Lewin et al., Nat. Biotechnol. 18:410-4, 2000; Josephson et al., Bioconjug., Chem. 10:186-91, 1999) suggesting that there is no apparent size restriction to this process.

The fusion of a protein transduction domain (PTD) with a heterologous molecule (e.g., a polynucleotide, small molecule, or protein) is sufficient to cause their transduction into a variety of different cells in a concentration-dependent manner. Moreover, this technique for protein delivery appears to circumvent many problems associated with DNA and drug based techniques.

PTDs are typically cationic in nature. These cationic protein transduction domains track into lipid raft endosomes carrying with them their linked cargo and release their cargo into the cytoplasm by disruption of the endosomal vesicle. Examples of PTDs include AntHD, TAT, VP22, cationic prion protein domains and functional fragments thereof. The disclosure provides methods and compositions that combine the use of PTDs such as TAT and poly-Arg, with a receptor ligand and a heterologous (e.g., “cargo”) domain. These compositions provide methods whereby a therapeutic or diagnostic agent can be selectively targeted to cells comprising a binding partner/cognate for the ligand and whereby the PTD causes uptake of the composition into the targeted cells.

In general, the transduction domain of the fusion molecule can be nearly any synthetic or naturally-occurring amino acid sequence that can transduce or assist in the transduction of the fusion molecule. For example, transduction can be achieved in accord with the invention by use of a protein sequence such as an HIV TAT protein or fragment thereof that is covalently linked at the N-terminal or C-terminal end to the ligand domain, the heterologous domain or both. Alternatively, the transducing protein can be the Antennapedia homeodomain or the HSV VP22 sequence, the N-terminal fragment of a prion protein or suitable transducing fragments thereof such as those known in the art.

The type and size of the PTD will be guided by several parameters including the extent of transduction desired. PTDs will be capable of transducing at least about 20%, 25%, 50%, 75%, 80% or 90% of the cells of interest, more preferably at least about 95%, 98% and up to, and including, about 100% of the cells. Transduction efficiency, typically expressed as the percentage of transduced cells, can be determined by several conventional methods.

PTDs will manifest cell entry and exit rates (sometimes referred to as k₁ and k₂, respectively) that favor at least picomolar amounts of the fusion molecule in the cell. The entry and exit rates of the PTD and any cargo can be readily determined or at least approximated by standard kinetic analysis using detectably-labeled fusion molecules. Typically, the ratio of the entry rate to the exit rate will be in the range of between about 5 to about 100 up to about 1000.

In one aspect, a PTD useful in the methods and compositions of the invention comprise a peptide featuring substantial alpha-helicity. It has been discovered that transduction is optimized when the PTD exhibits significant alpha-helicity. In another embodiment, the PTD comprises a sequence containing basic amino acid residues that are substantially aligned along at least one face of the peptide. A PTD domain of the invention may be a naturally occurring peptide or a synthetic peptide.

In another aspect of the invention, the PTD comprises an amino acid sequences comprising a strong alpha helical structure with arginine (Arg) residues down the helical cylinder.

In yet another embodiment, the PTD domain comprises a peptide represented by the following general formula: B1-X₁-X₂-X₃-B₂-X₄-X₅-B₃ (SEQ ID NO:1) wherein B₁, B₂, and B3 are each independently a basic amino acid, the same or different; and X₁, X₂, X₃, X₄ and X₅ are each independently an alpha-helix enhancing amino acid the same or different.

In another embodiment, the PTD domain is represented by the following general formula: B₁-X₁-X₂-B₂-B₃-X₃-X₄-B₄ (SEQ ID NO:2) wherein B₁, B₂, B₃, and B₄ are each independently a basic amino acid, the same or different; and X₁, X₂, X₃, and X₄ are each independently an alpha-helix enhancing amino acid the same or different.

Additionally PTD domains comprise basic residues, e.g., lysine (Lys) or arginine (Arg), and further including at least one proline (Pro) residue sufficient to introduce “kinks” into the domain. Examples of such domains include the transduction domains of prions. For example, such a peptide comprises KKRPKPG (SEQ ID NO:3).

In one embodiment, the domain is a peptide represented by the following sequence: X-X-R-X-(P/X)-(B/X)-B-(P/X)-X-B-(B/X) (SEQ ID NO:4), wherein X is any alpha helical promoting residue such as alanine; P/X is either proline or X as previously defined; B is a basic amino acid residue, e.g., arginine (Arg) or lysine (Lys); R is arginine (Arg) and B/X is either B or X as defined above.

In another embodiment the PTD is cationic and consists of between 7 and 10 amino acids and has the formula KX₁RX₂X₁ (SEQ ID NO:5) wherein X₁ is R or K and X₂ is any amino acid. An example of such a peptide comprises RKKRRQRRR (SEQ ID NO:6).

Additional transducing domains in accord with this invention include a TAT fragment that comprises at least amino acids 49 to 56 of TAT up to about the full-length TAT sequence (see, e.g., SEQ ID NO:7). A TAT fragment may include one or more amino acid changes sufficient to increase the alpha-helicity of the fragment. In some instances, the amino acid changes introduced will involve adding a recognized alpha-helix enhancing amino acid. Alternatively, the amino acid changes will involve removing one or more amino acids from the TAT fragment the impede alpha helix formation or stability. In a more specific embodiment, the TAT fragment will include at least one amino acid substitution with an alpha-helix enhancing amino acid. Typically the TAT fragment will be made by standard peptide synthesis techniques although recombinant DNA approaches may be used in some cases.

Additional transduction proteins (PTDs) that can be used in the compositions and methods of the invention include the TAT fragment in which the TAT 49-56 sequence has been modified so that at least two basic amino acids in the sequence are substantially aligned along at least one face of the TAT fragment. Illustrative TAT fragments include at least one specified amino acid substitution in at least amino acids 49-56 of TAT which substitution aligns the basic amino acid residues of the 49-56 sequence along at least one face of the segment and typically the TAT 49-56 sequence.

Additional transduction proteins in accord with this invention include the TAT fragment in which the TAT 49-56 sequence includes at least one substitution with an alpha-helix enhancing amino acid. In one embodiment, the substitution is selected so that at least two basic amino acid residues in the TAT fragment are substantially aligned along at least one face of that TAT fragment. In a more specific embodiment, the substitution is chosen so that at least two basic amino acid residues in the TAT 49-56 sequence are substantially aligned along at least one face of that sequence.

Also included are chimeric PTD domains. Such chimeric transducing proteins include parts of at least two different transducing proteins. For example, chimeric transducing proteins can be formed by fusing two different TAT fragments, e.g., one from HIV-1 and the other from HIV-2 or one from a prion protein and one from HIV.

PTDs can be linked or fused with any number of ligand domains. The ligand domains serve one or more purposes including, for example, to target the fusion polypeptide to a target cell expressing the ligand's cognate receptor and/or to promote uptake of the fusion polypeptide. Furthermore, the fusion polypeptide comprising the PTD and the ligand domain can be linked to any number of heterologous molecules having, for example, a therapeutic and/or diagnostic effect.

By the term “fusion polypeptide” as it is used herein is meant a transducing molecule such as a PTD protein or peptide sequence covalently linked (e.g., fused) to one or more heterologous polypeptides (e.g., a cytotoxic domain and a ligand domain) by recombinant, chemical or other suitable method. If desired, the fusion polypeptide can be fused at one or several sites through a peptide linker. The peptide linker can comprise one or more sites for cleavage by a pathogen induced or host cell induced protease. Alternatively, the peptide linker may be used to assist in construction of the fusion polypeptide or to assist in purification of the fusion polypeptide.

As noted, components of the fusion polypeptides disclosed herein, e.g., a PTD domain, a ligand domain, a heterologous domain, and optionally peptide linkers, can be organized in nearly any fashion provided that the fusion polypeptide has the function for which it was intended. The invention provides fusion polypeptides or chimeric proteins comprising one or more PTDs linked to a ligand domain which is either directly or indirectly linked to a heterologous domain (e.g., a therapeutic or diagnostic agent). Each of the several domains may be directly linked or may be separated by a linker peptide. The domains may be presented in any order (e.g., PTD-ligand-heterologous domain; ligand-PTD-heterologous domain; ligand-heterologous domain-PTD; heterologous domain-PTD-ligand; and similar variations). Additionally, the fusion polypeptides may include tags, e.g., to facilitate identification and/or purification of the fusion polypeptide, such as a 6×HIS tag.

Peptide linkers that can be used in the fusion polypeptides and methods of the invention will typically comprise up to about 20 or 30 amino acids, commonly up to about 10 or 15 amino acids, and still more often from about 1 to 5 amino acids. The linker sequence is generally flexible so as not to hold the fusion molecule in a single rigid conformation. The linker sequence can be used, e.g., to space the PTD domain from the ligand and/or heterologous domain. For example, the peptide linker sequence can be positioned between the protein transduction domain and the heterologous domain, e.g., to provide molecular flexibility. The length of the linker moiety is chosen to optimize the biological activity of the polypeptide comprising a PTD domain-ligand domain fusion and a heterologous molecule and can be determined empirically without undue experimentation. The linker moiety should be long enough and flexible enough to allow a ligand of the fusion construct to freely interact with its binding partner. Examples of linker moieties are -Gly-Gly-, GGGGS (SEQ ID NO:9), (GGGGS)_{N} (SEQ ID NO:10), GKSSGSGSESKS (SEQ ID NO:11), GSTSGSGKSSEGKG (SEQ ID NO:12), GSTSGSGKSSEGSGSTKG (SEQ ID NO:13), GSTSGSGKPGSGEGSTKG (SEQ ID NO:14), or EGKSSGSGSESKEF (SEQ ID NO:15). Linking moieties are described, for example, in Huston et al., Proc. Nat'l Acad. Sci. 85:5879, 1988; Whitlow et al., Protein Engineering 6:989, 1993; and Newton et al., Biochemistry 35:545, 1996. Other suitable peptide linkers are those described in U.S. Pat. Nos. 4,751,180 and 4,935,233, which are hereby incorporated by reference.

The methods, compositions, and fusion polypeptides of the invention provide enhanced uptake and release of PTDs linked to heterologous molecules. A PTD fusion polypeptide can comprise a PTD domain, a receptor ligand; and a heterologous domain with or without additional domains (e.g., fusogenic domains).

As used herein, a “fusogenic” domain is any polypeptide that facilitates the destabilization of a cell membrane or the membrane of a cell organelle. For example, the hemagglutinin (HA) of influenza is the major glycoprotein component of the viral envelope. It has a dual function in mediating attachment of the virus to the target cell and fusion of the viral envelope membrane with target cell membranes. In the normal course of viral infection, virus bound to the cell surface is taken up into endosomes and exposed to relatively low pH. The pH change triggers fusion between the viral envelope and the endosomal membrane, as well as conformational changes in HA, which lead to increased exposure of the amino terminus. Synthetic peptides such as the N-terminus region of the influenza hemagglutinin protein destabilize membranes. Examples of HA2 analogs include GLFGAIAGFIEGGWTGMIDG (SEQ ID NO: 15) and GLFEAIAEFIEGGWEGLIEG (SEQ ID NO: 16).

Other fusogenic proteins include, for example, the M2 protein of influenza A viruses employed on its own or in combination with the hemagglutinin of influenza virus or with mutants of neuraminidase of influenza A, which lack enzyme activity, but which bring about hemagglutination; peptide analogs of the influenza virus hemagglutinin; the HEF protein of the influenza C virus, the fusion activity of the HEF protein is activated by cleavage of the HEFo into the subunits HEF1 and HEF2; the transmembrane glycoprotein of filoviruses, such as, for example, the Marburg virus, the Ebola virus; the transmembrane glycoprotein of the rabies virus; the transmembrane glycoprotein (G) of the vesicular stomatitis virus; the fusion polypeptide of the Sendai virus, in particular the amino-terminal 33 amino acids of the F1 component; the transmembrane glycoprotein of the Semliki forest virus, in particular the E1 component, the transmembrane glycoprotein of the tickborn encephalitis virus; the fusion polypeptide of the human respiratory syncytial virus (RSV) (in particular the gp37 component); the fusion polypeptide (S protein) of the hepatitis B virus; the fusion polypeptide of the measles virus; the fusion polypeptide of the Newcastle disease virus; the fusion polypeptide of the visna virus; the fusion polypeptide of murine leukemia virus (in particular p15E); the fusion polypeptide of the HTL virus (in particular gp21); and the fusion polypeptide of the simian immunodeficiency virus (SIV). Viral fusogenic proteins are obtained either by dissolving the coat proteins of a virus concentration with the aid of detergents (such as, for example, β-D-octylglucopyranoside) and separation by centrifugation (review in Mannio et al., BioTechniques 6, 682 (1988)) or else with the aid of molecular biology methods known to the person skilled in the art.

A transducible PTD-ligand domain-fusogenic fusion polypeptide (e.g., HA2-TAT-DV3 fusion polypeptide) enhances release of heterologous molecules from the endosome into the cytoplasm, nucleus or other cellular organelle. This is accomplished by the PTD-ligand domain-fusogenic fusion polypeptide tracking with the PTD-ligand domain-heterologous fusion polypeptide via independent or the same PTD domain and receptor ligand and then fusing to the vesicle lipid bilayer by the fusogenic domain (e.g., HA2) resulting in an enhanced release into the cytoplasm, nucleus, or other cellular organelle. Thus, the disclosure provides a transduction domain (PTD) associated with a ligand and a heterologous domain; and a transduction domain (PTD) associated with a receptor ligand (the same or different) and a fusogenic (i.e., to facilitate membrane fusion) domain. For example, a PTD associated with a receptor ligand and a heterologous molecule can comprise a single chimeric/fusion polypeptide. Similarly, a PTD associated with a receptor ligand and a fusogenic domain can comprise a single chimeric/fusion polypeptide. The fusion of functionally distinguishable domains to generate chimeric/fusion polypeptides is known in the art.

The ability of PTDs to transducer heterologous (i.e., cargo) domains into cells have been successfully demonstrated in vitro and in vivo. Examples of PTDs fused with various heterologous domains is provided in Table 1. These applications cover a broad range of uses and, in general, there appears to be no particular limitation in either the size or type of protein that can be delivered. TAT protein transduction has been useful in a variety of situations to overcome the limitations of traditional DNA-based approaches or for the development of novel strategies in the treatment of disease.

TABLE 1

| TAT-Protein | Effect | References |
|---|---|---|
| TAT-Bcl-xL | anti-apoptotic | Cao et al., (2002) J. |
| | | Neurosci. 22, 5423-31, |
| | | Kilic et al., (2002) Ann. |
| | | Neurol. 52, 617-22, |
| | | Dietz et al., (2002) Mol. |
| | | Cell Neurosci. 21, 29-37, |
| | | Embury et al., (2001) |
| | | Diabetes 50, 1706-13 |
| TAT-p53 | tumor suppressor | Takenobu et al., (2002) |
| | protein | Mol. Cancer Ther. 1, |
| | | 1043-9 |
| TAT-ARC | transduction into | Gustafsson et al., (2002) |
| | myocardium is | Circulation 106, 735-9 |
| | cardioprotective | |
| TAT-cyclin | E restoration of | Hsia et al., (2002) Int. |
| | proliferation | Immunol. 14, 905-16 |
| TAT-glutamate | restoration of | Yoon et al., (2002) |
| dehydrogenase | GDH-deficiency | Neurochem. Int. 41, 37- |
| | disorders | 42 |
| TAT-Cu, Zn-SOD | antioxidant protein | Kwon et al., (2000) |
| | | FEBS Lett. 485, 163-7, |
| | | Eum et al., (2002) Mol. |
| | | Cells 13, 334-40 |
| TAT-catalase | antioxidant protein | Jin et al., (2001) Free |
| | | Radic. Biol. Med. 31, |
| | | 1509-19 |
| TAT-ODD- | anti-tumor activity | Harada et al., 2002) |
| Caspase 3 | | Cancer Res. 62, 2013-8 |
| TAT-HIV1- | specific killing of | Vocero-Akbani et al., |
| Caspase 3 | HIV-infected cells | (1999) Nat. Med. 5, 29- |
| | | 33 |
| TAT-Cre | site-specific | Joshi et al., (2002) |
| | recombination | Genesis. 33, 48-54, Peitz |
| | | et al., (2002) Proc. Natl. |
| | | Acad. Sci. USA 99, |
| | | 4489-94 |
| TAT-APOBEC | editing of ApoB mRNA | Yang et al., (2002) Mol. |
| | | Pharmacol. 61, 269-76 |
| TAT-GFP | fluorescent protein | Caron et al., (2001) Mol. |
| | | Ther. 3, 310-8, Han et |
| | | al., (2001) Mol. Cells 12, |
| | | 267-71 |
| TAT-H-Ras | cytoskeletal | Hall et al., (2001) Blood |
| | reorganization | 98, 2014-21 |
| TAT-IkappaB | NF-kappaB | Abu-Amer et al., 2001) |
| | inhibitory protein | J. Biol. Chem. 276, |
| | | 30499-503. |
| TAT-HPC-1/ | inhibitor of | Fujiwara et al., (2001) |
| syntaxin | neurotransmitter | Biochim. Biophys. Acta |
| | release | 1539, 225-32 |
| TAT-p16 | inhibitor of cyclin | Ezhevsky et al., (2001) |
| | D/cdk complexes | Mol. Cell Biol. 21, 4773- |
| | | 84 |
| TAT-p27 | cyclin-dependent | McAllister et al., (2003) |
| | kinase inhibitor | Mol. Cell Biol. 23, 216- |
| | | 28 |
| TAT-b- | frequently used | Barka et al., (2000) J. |
| galactosidase | reporter enzyme | Histochem. Cytochem. |
| | | 48, 1453-1460, |
| | | Schwarze et al., (1999) |
| | | Science 285, 1569-72 |
| TAT-p21 | cell cycle arrest | Kunieda et al., (2002) |
| | in G1 phase | Cell Transplant 11, 421- |
| | | 8 |
| TAT-PEA-15 | prevents apoptosis | Embury et al., (2001) |
| | by TNFa in pancreatic | Diabetes 50, 1706-13 |
| | cell line | |
| TAT-beta- | lysosomal enzyme | Xia et al., (2001) Nat. |
| glucuronidase | | Biotechnol. 19, 640-4 |

The invention provides methods, compositions, and fusion polypeptides that target specific cells (e.g., cells having a particular phenotype characteristic comprising, for example, specific cell surface receptors) using ligand domains.

A ligand domain (e.g., a targeting molecule) for use in the invention includes, but is not limited to, a ligand or an antibody that specifically binds to its corresponding target, for example, a receptor on a cell surface. Thus, for example, where the ligand domain is an antibody, the fusion polypeptide will specifically bind (target) cells and tissues bearing the epitope to which the antibody is directed. Thus, a ligand refers generally to all molecules capable of reacting with or otherwise recognizing or binding to a receptor or polypeptide on a target cell. Any known ligand or targeting molecule can be used as the ligand domain of the fusion polypeptide of the invention. Examples of targeting peptides that can be manipulated and cloned or linked to produce a fusion polypeptide are ample in the literature. In general, any peptide ligand can be used or fragments thereof based on the receptor-binding sequence of the ligand. In immunology, such a peptide domain is referred to as an epitope, and the term epitope may be used herein to refer to a ligand recognized by a receptor. For example, a ligand comprises the sequence of a protein or peptide that is recognized by a binding partner on the surface of a target cell, which for the sake of convenience is termed a receptor. However, it should be understood that for purposes of the invention, the term “receptor” encompasses signal-transducing receptors (e.g., receptors for hormones, steroids, cytokines, insulin, and other growth factors), recognition molecules (e.g., MHC molecules, B- or T-cell receptors), nutrient uptake receptors (such as transferrin receptor), lectins, ion channels, adhesion molecules, extracellular matrix binding proteins, and the like that are located and accessible at the surface of the target cell.

A number of chemokine ligands are known in the art. For example, DV3 is used in the Examples herein; however other chemokine ligands are known in the art (see, e.g., Zhou et al., J. Biol. Chem., 277(20):17476-17485, 2002, incorporated herein by reference).

The size of the ligand domain peptide can vary within certain parameters. Examples of ligands include, but are not limited to, antibodies, lymphokines, cytokines, receptor proteins such as CD4 and CD8, hormones, growth factors, and the like which specifically bind desired target cells. For example, several human malignancies overexpress specific receptors, including HER2, LHRH and CXCR4. Accordingly, ligands to these receptors can be used in the fusion polypeptides, methods and compositions of the invention. Receptor ligand domains are known in the art.

The heterologous domain (i.e., cargo domain) of the fusion polypeptide of the invention can comprise a therapeutic agent and/or a diagnostic agent. Examples of selected agents include therapeutic agents, such as thrombolytic agents and anticellular agents that kill or suppress the growth or cell division of disease-associated cells (e.g., cells comprising a cell proliferative disorder such as a neoplasm or cancer). Examples of effective thrombolytic agents are streptokinase and urokinase.

Effective anticellular agents include classical chemotherapeutic agents, such as steroids, antimetabolites, anthracycline, vinca alkaloids, antibiotics, alkylating agents, epipodophyllotoxin and anti-tumor agents such as neocarzinostatin (NCS), adriamycin and dideoxycytidine; mammalian cell cytotoxins, such as interferon-α (IFN-α), interferon-βγ (IFN-βγ), interleukin-12 (IL-12) and tumor necrosis factor-α (TNF-α); plant-, fungus- and bacteria-derived toxins, such as ribosome inactivating protein, gelonin, α-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain; as well as radioisotopes.

Diagnostic agents will generally be a fluorogenic, paramagnetic or radioactive ion that is detectable upon imaging. Examples of paramagnetic ions include chromium (III), manganese (II), iron (III), iron (II), cobalt (I), nickel (II), copper (II), neodymium (III), samarium (III), ytterbium (II), gadolinium (III), vanadium (II), terbium (III), dysprosium (III), holmium (III) and erbium (III) ions.

Examples of radioactive ions include iodine¹²³, technicium^{99m}, indium¹¹¹, rhenium¹⁸⁸, rhenium¹⁸⁶, copper⁶⁷, iodine¹³¹, yttrium⁹⁰, iodine¹²⁵, astatine²¹¹, gallium⁶⁷, iridium¹⁹², cobalt⁶⁰, radium²²⁶, gold¹⁹⁸, cesium¹³⁷ and phosphorus³² ions. Examples of fluorogenic agents include gadolinium and renographin.

In attaching a fluorogenic, paramagnetic or radioactive ion to a fusion polypeptide of the invention, the agent is linked to the protein or polypeptide carrier, using methods commonly known in the art.

As used herein, a heterologous domain can be (1) any heterologous polypeptide, or fragment thereof, (2) any polynucleotide (e.g., a ribozyme, antisense molecule, polynucleotide, oligonucleotide and the like); (3) any small molecule, or (4) any diagnostic or therapeutic agent, that is capable of being linked or fused to protein backbone (e.g., linked or fused to a PTD or ligand domain). For example, PTD fusion molecule can comprise a PTD-ligand domain linked to a heterologous polypeptide, or fragment thereof, that provides a therapeutic effect when present in a targeted cell.

The term “therapeutic” is used in a generic sense and includes treating agents, prophylactic agents, and replacement agents. Examples of therapeutic molecules include, but are not limited to, cell cycle control agents; agents which inhibit cyclin proteins, such as antisense polynucleotides to the cyclin G1 and cyclin D1 genes; growth factors such as, for example, epidermal growth factor (EGF), vascular endothelial growth factor (VEGF), erythropoietin, G-CSF, GM-CSF, TGF-α, TGF-β, and fibroblast growth factor; cytokines, including, but not limited to, Interleukins 1 through 13 and tumor necrosis factors; anticoagulants, anti-platelet agents; anti-inflammatory agents (e.g., soluble TNF receptor domains such as ENBREL); tumor suppressor proteins; clotting factors including Factor VIII and Factor IX, protein S, protein C, antithrombin III, von Willebrand Factor, cystic fibrosis transmembrane conductance regulator (CFTR), and negative selective markers such as Herpes Simplex Virus thymidine kinase.

In addition, a heterologous molecule fused to the PTD-ligand domain can be a negative selective marker or “suicide” protein, such as, for example, the Herpes Simplex Virus thymidine kinase (TK). Such a PTD linked to a suicide protein may be administered to a subject whereby tumor cells are selectively transduced. After the tumor cells are transduced with the kinase, an interaction agent, such as gancyclovir or acyclovir, is administered to the subject, whereby the transduced tumor cells are killed. Growth of the tumor cells is inhibited, suppressed, or destroyed upon expression of the anti-tumor agent by the transduced tumor cells.

In addition, a heterologous molecule can be a diagnostic agent such as an imaging agent. For example, a PTD-ligand fusion polypeptide can be fused to a radio-labeled moiety.

Thus, it is to be understood that the disclosure is not to be limited to any particular heterologous domain used for diagnosis and/or treatment of any particular disease or disorder. Rather, the heterologous domain can be any domain known or used in other fusion proteins in the art for treatment or delivery of diagnostic or therapeutic agents.

The polypeptides used in the invention (e.g., with respect to particular domains of a fusion polypeptide or the full length fusion polypeptide) can comprise either the L-optical isomer or the D-optical isomer of amino acids or a combination of both. Polypeptides that can be used in the invention include modified sequences such as glycoproteins, retro-inverso polypeptides, D-amino acid modified polypeptides, and the like. A polypeptide includes naturally occurring proteins, as well as those which are recombinantly or synthetically synthesized. “Fragments” are a portion of a polypeptide. The term “fragment” refers to a portion of a polypeptide which exhibits at least one useful epitope or functional domain. The term “functional fragment” refers to fragments of a polypeptide that retain an activity of the polypeptide. For example, a functional fragment of a PTD includes a fragment which retains transduction activity. Biologically functional fragments, for example, can vary in size from a polypeptide fragment as small as an epitope capable of binding an antibody molecule, to a large polypeptide capable of participating in the characteristic induction or programming of phenotypic changes within a cell. An “epitope” is a region of a polypeptide capable of binding an immunoglobulin generated in response to contact with an antigen. Small epitopes of receptor ligands can be useful in the methods of the invention so long as it retains the ability to interact with the receptor.

In some embodiments, retro-inverso peptides are used. “Retro-inverso” means an amino-carboxy inversion as well as enantiomeric change in one or more amino acids (i.e., levantory (L) to dextrorotary (D)). A polypeptide of the disclosure encompasses, for example, amino-carboxy inversions of the amino acid sequence, amino-carboxy inversions containing one or more D-amino acids, and non-inverted sequence containing one or more D-amino acids. Retro-inverso peptidomimetics that are stable and retain bioactivity can be devised as described by Brugidou et al. (Biochem. Biophys. Res. Comm. 214(2): 685-693, 1995) and Chorev et al. (Trends Biotechnol. 13(10): 438-445, 1995).

In another aspect, the disclosure provides a method of producing a fusion polypeptide comprising a PTD domain, a ligand domain and a heterologous molecule or a fusogenic domain by growing a host cell comprising a polynucleotide encoding the fusion polypeptide under conditions that allow expression of the polynucleotide, and recovering the fusion polypeptide. A polynucleotide encoding a fusion polypeptide of the disclosure can be operably linked to a promoter for expression in a prokaryotic or eukaryotic expression system. For example, such a polynucleotide can be incorporated in an expression vector.

Accordingly, the invention also provides polynucleotides encoding a fusion protein construct of the invention. Such polynucleotides comprise sequences encoding a PTD domain, a ligand domain, and a heterologous domain operably linked in any order. The polynucleotide may also encode linker domains that separate one or more of the PTD, ligand and heterologous domains.

Delivery of a polynucleotide of the disclosure can be achieved by introducing the polynucleotide into a cell using a variety of methods known to those of skill in the art. For example, a construct comprising such a polynucleotide can be delivered into a cell using a colloidal dispersion system. Alternatively, a polynucleotide construct can be incorporated (i.e., cloned) into an appropriate vector. For purposes of expression, the polynucleotide encoding a fusion polypeptide of the disclosure may be inserted into a recombinant expression vector. The term “recombinant expression vector” refers to a plasmid, virus, or other vehicle known in the art that has been manipulated by insertion or incorporation of a polynucleotide encoding a fusion polypeptide of the disclosure. The expression vector typically contains an origin of replication, a promoter, as well as specific genes that allow phenotypic selection of the transformed cells. Vectors suitable for such use include, but are not limited to, the T7-based expression vector for expression in bacteria (Rosenberg et al., Gene, 56:125, 1987), the pMSXND expression vector for expression in mammalian cells (Lee and Nathans, J. Biol. Chem., 263:3521, 1988), baculovirus-derived vectors for expression in insect cells, cauliflower mosaic virus, CaMV, and tobacco mosaic virus, TMV, for expression in plants.

Depending on the vector utilized, any of a number of suitable transcription and translation elements (regulatory sequences), including constitutive and inducible promoters, transcription enhancer elements, transcription terminators, and the like may be used in the expression vector (see, e.g., Bitter et al., Methods in Enzymology, 153:516-544, 1987). These elements are well known to one of skill in the art.

The term “operably linked” or “operably associated” refers to functional linkage between a regulatory sequence and the polynucleotide regulated by the regulatory sequence as well as the link between encoded domains of the fusion polypeptides such that each domain is linked in-frame to give rise to the desired polypeptide sequence.

In yeast, a number of vectors containing constitutive or inducible promoters may be used (see, e.g., Current Protocols in Molecular Biology, Vol. 2, Ed. Ausubel et al., Greene Publish. Assoc. & Wiley Interscience, Ch. 13, 1988; Grant et al., “Expression and Secretion Vectors for Yeast,” in Methods in Enzymology, Eds. Wu & Grossman, Acad. Press, N.Y, Vol. 153, pp. 516-544, 1987; Glover, DNA Cloning, Vol. II, IRL Press, Wash., D.C., Ch. 3, 1986; “Bitter, Heterologous Gene Expression in Yeast,” Methods in Enzymology, Eds. Berger & Kimmel, Acad. Press, N.Y, Vol. 152, pp. 673-684, 1987; and The Molecular Biology of the Yeast Saccharomyces, Eds. Strathern et al., Cold Spring Harbor Press, Vols. 1 and 1, 1982). A constitutive yeast promoter, such as ADH or LEU2, or an inducible promoter, such as GAL, may be used (“Cloning in Yeast,” Ch. 3, R. Rothstein In: DNA Cloning Vol. 11, A Practical Approach, Ed. D M Glover, IRL Press, Wash., D.C., 1986). Alternatively, vectors may be used which promote integration of foreign DNA sequences into the yeast chromosome.

An expression vector can be used to transform a host cell. By “transformation” is meant a permanent genetic change induced in a cell following incorporation of a polynucleotide exogenous to the cell. Where the cell is a mammalian cell, a permanent genetic change is generally achieved by introduction of the polynucleotide into the genome of the cell. By “transformed cell” or “recombinant host cell” is meant a cell into which (or into an ancestor of which) has been introduced, by means of molecular biology techniques, a polynucleotide encoding a fusion polypeptide of the invention. Transformation of a host cell may be carried out by conventional techniques as are known to those skilled in the art. Where the host is prokaryotic, such as E. coli, competent cells which are capable of polynucleotide uptake can be prepared from cells harvested after exponential growth phase and subsequently treated by the CaCl₂ method by procedures known in the art. Alternatively, MgCl₂ or RbCl can be used. Transformation can also be performed after forming a protoplast of the host cell or by electroporation.

A fusion polypeptide of the disclosure can be produced by expression of polynucleotide encoding a fusion polypeptide in prokaryotes. These include, but are not limited to, microorganisms, such as bacteria transformed with recombinant bacteriophage DNA, plasmid DNA, or cosmid DNA expression vectors encoding a fusion polypeptide of the disclosure. The constructs can be expressed in E. coli in large scale. Purification from bacteria is simplified when the sequences include tags for one-step purification by nickel-chelate chromatography. Thus, a polynucleotide encoding a fusion polypeptide can also comprise a tag to simplify isolation of the fusion polypeptide. For example, a polyhistidine tag of, e.g., six histidine residues, can be incorporated at the amino terminal end of the fusion polypeptide. The polyhistidine tag allows convenient isolation of the protein in a single step by nickel-chelate chromatography. A fusion polypeptide of the disclosure can also be engineered to contain a cleavage site to aid in protein recovery the cleavage site may be part of a linker moiety as discussed above. A DNA sequence encoding a desired peptide linker can be inserted between, and in the same reading frame as, a polynucleotide encoding a PTD, or fragment thereof followed by a receptor ligand and followed by a heterologous polypeptide, using any suitable conventional technique. For example, a chemically synthesized oligonucleotide encoding the linker can be ligated between two coding polynucleotides. In particular embodiments, a polynucleotide of the invention will encode a fusion polypeptide comprising from three to four separate domains (e.g., a PTD domain, a receptor ligand domain and a heterologous polypeptide domain) are separated by peptide linkers.

When the host cell is a eukaryotic cell, such methods of transfection of DNA as calcium phosphate co-precipitates, conventional mechanical procedures, such as microinjection, electroporation, insertion of a plasmid encased in liposomes, or virus vectors may be used. Eukaryotic cells can also be cotransfected with a polynucleotide encoding the PTD-fusion polypeptide of the disclosure, and a second polynucleotide molecule encoding a selectable phenotype, such as the herpes simplex thymidine kinase gene. Another method is to use a eukaryotic viral vector, such as simian virus 40 (SV40) or bovine papilloma virus, to transiently infect or transform eukaryotic cells and express the fusion polypeptide (see, e.g., Eukaryotic Viral Vectors, Cold Spring Harbor Laboratory, Gluzman ed., 1982).

Eukaryotic systems, and typically mammalian expression systems, allow for proper post-translational modifications of expressed mammalian proteins to occur. Eukaryotic cells that possess the cellular machinery for proper processing of the primary transcript, glycosylation, phosphorylation, and advantageously secretion of the fusion product can be used as host cells for the expression of the PTD-fusion polypeptide of the disclosure. Such host cell lines may include, but are not limited to, CHO, VERO, BHK, HeLa, COS, MDCK, Jurkat, HEK-293, and WI38.

For long-term, high-yield production of recombinant proteins, stable expression is used. Rather than using expression vectors that contain viral origins of replication, host cells can be transformed with the cDNA encoding a fusion polypeptide of the disclosure controlled by appropriate expression control elements (e.g., promoter, enhancer, sequences, transcription terminators, polyadenylation sites, and the like), and a selectable marker. The selectable marker in the recombinant plasmid confers selectivity (e.g., by cytotoxin resistance) and allows cells to stably integrate the plasmid into their chromosomes and grow to form foci that, in turn, can be cloned and expanded into cell lines. For example, following the introduction of foreign DNA, engineered cells may be allowed to grow for 1-2 days in an enriched media, and then are switched to a selective media. A number of selection systems may be used, including, but not limited to, the herpes simplex virus thymidine kinase (Wigler et al., Cell, 11:223, 1977), hypoxanthine-guanine phosphoribosyltransferase (Szybalska & Szybalski, Proc. Natl. Acad. Sci. USA, 48:2026, 1962), and adenine phosphoribosyltransferase (Lowy et al., Cell, 22:817, 1980) genes can be employed in tk-, hgprt- or aprt-cells, respectively. Also, antimetabolite resistance can be used as the basis of selection for dhfr, which confers resistance to methotrexate (Wigler et al., Proc. Natl. Acad. Sci. USA, 77:3567, 1980; O'Hare et al., Proc. Natl. Acad. Sci. USA, 8:1527, 1981); gpt, which confers resistance to mycophenolic acid (Mulligan & Berg, Proc. Natl. Acad. Sci. USA, 78:2072, 1981; neo, which confers resistance to the aminoglycoside G-418 (Colberre-Garapin et al., J. Mol. Biol., 150:1, 1981); and hygro, which confers resistance to hygromycin genes (Santerre et al., Gene, 30:147, 1984). Additional selectable genes have been described, namely trpB, which allows cells to utilize indole in place of tryptophan; hisD, which allows cells to utilize histinol in place of histidine (Hartman & Mulligan, Proc. Natl. Acad. Sci. USA, 85:8047, 1988); and ODC (ornithine decarboxylase), which confers resistance to the ornithine decarboxylase inhibitor, 2-(difluoromethyl)-DL-ornithine, DFMO (McConlogue L., In: Current Communications in Molecular Biology, Cold Spring Harbor Laboratory, ed., 1987).

Techniques for the isolation and purification of either microbially or eukaryotically expressed PTD-fusion polypeptides of the disclosure may be by any conventional means, such as, for example, preparative chromatographic separations and immunological separations, such as those involving the use of monoclonal or polyclonal antibodies or antigen.

The fusion polypeptides of the invention are useful for the treatment and/or diagnosis of a number of diseases and disorders. For example, the fusion polypeptides can be used in the treatment of cell proliferative disorders, wherein the ligand domain targets the fusion polypeptide to a target binding domain on a cell-type of interest and wherein the heterologous domain comprises a cytotoxic agent. The PTD domain facilitates uptake of the fusion polypeptide and the ligand domain facilitates cell-specific targeting. Thus, the fusion polypeptide is useful for treatment and selective targeting of cells having cell proliferative disorders. Similarly, the fusion polypeptides of the invention can be used to treatment inflammatory diseases and disorders, infections, vascular disease and disorders and the like.

Typically a fusion polypeptide of the invention will be formulated with a pharmaceutically acceptable carrier, although the fusion polypeptide may be administered alone, as a pharmaceutical composition.

A pharmaceutical composition according to the disclosure can be prepared to include a fusion polypeptide of the disclosure, into a form suitable for administration to a subject using carriers, excipients, and additives or auxiliaries. Frequently used carriers or auxiliaries include magnesium carbonate, titanium dioxide, lactose, mannitol and other sugars, talc, milk protein, gelatin, starch, vitamins, cellulose and its derivatives, animal and vegetable oils, polyethylene glycols and solvents, such as sterile water, alcohols, glycerol, and polyhydric alcohols. Intravenous vehicles include fluid and nutrient replenishers. Preservatives include antimicrobial, anti-oxidants, chelating agents, and inert gases. Other pharmaceutically acceptable carriers include aqueous solutions, non-toxic excipients, including salts, preservatives, buffers and the like, as described, for instance, in Remington's Pharmaceutical Sciences, 15th ed., Easton: Mack Publishing Co., 1405-1412, 1461-1487 (1975), and The National Formulary XIV., 14th ed., Washington: American Pharmaceutical Association (1975), the contents of which are hereby incorporated by reference. The pH and exact concentration of the various components of the pharmaceutical composition are adjusted according to routine skills in the art. See Goodman and Gilman's, The Pharmacological Basis for Therapeutics (7th ed.).

The pharmaceutical compositions according to the disclosure may be administered locally or systemically. By “therapeutically effective dose” is meant the quantity of a fusion polypeptide according to the disclosure necessary to prevent, to cure, or at least partially arrest the symptoms of a disease or disorder (e.g., to inhibit cellular proliferation). Amounts effective for this use will, of course, depend on the severity of the disease and the weight and general state of the subject. Typically, dosages used in vitro may provide useful guidance in the amounts useful for in situ administration of the pharmaceutical composition, and animal models may be used to determine effective dosages for treatment of particular disorders. Various considerations are described, e.g., in Langer, Science, 249: 1527, (1990); Gilman et al. (eds.) (1990), each of which is herein incorporated by reference.

As used herein, “administering a therapeutically effective amount” is intended to include methods of giving or applying a pharmaceutical composition of the disclosure to a subject that allow the composition to perform its intended therapeutic function. The therapeutically effective amounts will vary according to factors, such as the degree of infection in a subject, the age, sex, and weight of the individual. Dosage regima can be adjusted to provide the optimum therapeutic response. For example, several divided doses can be administered daily or the dose can be proportionally reduced as indicated by the exigencies of the therapeutic situation.

The pharmaceutical composition can be administered in a convenient manner, such as by injection (e.g, subcutaneous, intravenous, and the like), oral administration, inhalation, transdermal application, or rectal administration. Depending on the route of administration, the pharmaceutical composition can be coated with a material to protect the pharmaceutical composition from the action of enzymes, acids, and other natural conditions that may inactivate the pharmaceutical composition. The pharmaceutical composition can also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersions. The composition will typically be sterile and fluid to the extent that easy syringability exists. Typically the composition will be stable under the conditions of manufacture and storage and preserved against the contaminating action of microorganisms, such as bacteria and fungi. The carrier can be a solvent or dispersion medium containing, for example, water, ethanol, polyol (for example, glycerol, propylene glycol, and liquid polyetheylene glycol, and the like), suitable mixtures thereof, and vegetable oils. The proper fluidity can be maintained, for example, by the use of a coating, such as lecithin, by the maintenance of the required particle size, in the case of dispersion, and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents, for example, sugars, polyalcohols, such as mannitol, sorbitol, or sodium chloride are used in the composition. Prolonged absorption of the injectable compositions can be brought about by including in the composition an agent that delays absorption, for example, aluminum monostearate and gelatin.

Sterile injectable solutions can be prepared by incorporating the pharmaceutical composition in the required amount in an appropriate solvent with one or a combination of ingredients enumerated above, as required, followed by filtered sterilization. Generally, dispersions are prepared by incorporating the pharmaceutical composition into a sterile vehicle that contains a basic dispersion medium and the required other ingredients from those enumerated above.

The pharmaceutical composition can be orally administered, for example, with an inert diluent or an assimilable edible carrier. The pharmaceutical composition and other ingredients can also be enclosed in a hard or soft-shell gelatin capsule, compressed into tablets, or incorporated directly into the subject's diet. For oral therapeutic administration, the pharmaceutical composition can be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations can, of course, be varied and can conveniently be between about 5% to about 80% of the weight of the unit.

The tablets, troches, pills, capsules, and the like can also contain the following: a binder, such as gum gragacanth, acacia, corn starch, or gelatin; excipients such as dicalcium phosphate; a disintegrating agent, such as corn starch, potato starch, alginic acid, and the like; a lubricant, such as magnesium stearate; and a sweetening agent, such as sucrose, lactose or saccharin, or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar, or both. A syrup or elixir can contain the agent, sucrose as a sweetening agent, methyl and propylparabens as preservatives, a dye, and flavoring, such as cherry or orange flavor. Of course, any material used in preparing any dosage unit form should be pharmaceutically pure and substantially non-toxic in the amounts employed. In addition, the pharmaceutical composition can be incorporated into sustained-release preparations and formulations.

Thus, a “pharmaceutically acceptable carrier” is intended to include solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the pharmaceutical composition, use thereof in the therapeutic compositions and methods of treatment is contemplated. Supplementary active compounds can also be incorporated into the compositions.

It is especially advantageous to formulate parenteral compositions in dosage unit form for ease of administration and uniformity of dosage. “Dosage unit form” as used herein, refers to physically discrete units suited as unitary dosages for the subject to be treated; each unit containing a predetermined quantity of pharmaceutical composition is calculated to produce the desired therapeutic effect in association with the required pharmaceutical carrier. The specification for the dosage unit forms of the disclosure are related to the characteristics of the pharmaceutical composition and the particular therapeutic effect to be achieve.

The principal pharmaceutical composition is compounded for convenient and effective administration in effective amounts with a suitable pharmaceutically acceptable carrier in an acceptable dosage unit. In the case of compositions containing supplementary active ingredients, the dosages are determined by reference to the usual dose and manner of administration of the said ingredients.

The working examples below are provided to illustrate, not limit, the invention. Various parameters of the scientific methods employed in these examples are described in detail below and provide guidance for practicing the invention in general.

### EXAMPLES

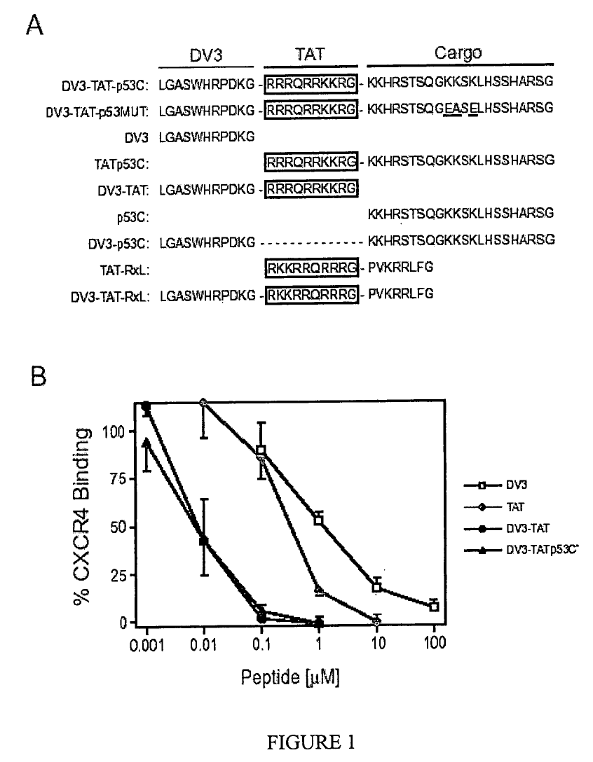

To test the hypothesis that delivery of PTDs could be selectively enhanced to tumor cells by targeting overexpressed receptors, a CXCR4 receptor ligand, DV3 was linked to two proven transducible anticancer peptides, a p53-activating peptide (TATp53C′) and a cdk2 antagonist peptide (TAT-RxL). The CXCR4 receptor DV3 ligand was linked to the N-terminus of a retroinverso, D-isomer transducible TATp53-activating peptide yielding DV3-TATp53C′ and mutant non-p53-activating, DV3-TATp53MUT_peptide (FIG. 1A). In addition, a previously characterized cdk2 antagonist peptide (TAT-RxL) and a DV3-TAT-RxL peptide version were generated as well as multiple control peptides (FIG. 1A).

The effect of these multi-domain, biologically active, macromolecular peptides (termed DV3-TATp53 and DV3-TAT-RxL) were observed on cancer cells that overexpress the CXCR4 receptor. Treatment of tumor cells overexpressing the CXCR4 receptor with DV3 targeted transducible anti-cancer peptides resulted in a dramatic enhancement of tumor cell killing in vitro and in vivo, compared to treatment with non-targeted parental peptides. In contrast, there was no difference between DV3 targeted peptide and non-targeted, parental peptide in non-CXCR4 expressing tumor cells. These observations demonstrate that a multi-domain approach can further enhance tumor selectivity of biologically active, transducible macromolecules for treating cancer.

Cell Culture and Flow Cytometry. TA3/St, H1299 and 293T cells were maintained in DMEM plus 10% fetal bovine serum (FBS) and penicillin/streptomycin (P/S). Namalwa B cells (ATCC) were maintained in RPMI plus 10% FBS, P/S. All cells were maintained at 37° C. in 5% CO₂. Short-term cell viability was assessed by counting Trypan blue excluding cells on a hemocytometer. For cell cycle analysis, peptide treated cells were analyzed by FACS with 10 μg/ml propidium iodide in 0.5% NP-40. DNA profiles were analyzed using a FACScan and CellQuest software (Becton Dickinson, San Jose, Calif.).

Apoptosis was determined by nuclei condensation of DAPI stained cells and microscopy.

Peptide Synthesis. D- and L-isomer peptides were synthesized by standard Fmoc chemistry on an ABI 433A Peptide Synthesizer (Applied Biosystems, Foster City, Calif.). Crude peptides were purified over a C18 HPLC preparatory column (Varian, Palo Alto, Calif.) and confirmed by mass spectrometry. DV3-TATp53C′, TATp53C′, DV3-TAT, DV3-p53c′, p53C′, and DV3 peptides were synthesized with Disomer residues, whereas the DV3 domain of DV3-TAT-RxL was D-isomer residues and the TAT-RxL domain was L-isomer residues.

CXCR4 binding assay. CXCR4 expressing Namalwa cells were washed 3 times with PBS/0.5% BSA and incubated on ice with phycoerythrin (PE)-labeled anti-CXCR4 monoclonal antibody (12G5-PE, R&D Systems, WI) and peptide. PE-labeled isotype matched antibody was used to control for non-specific cell surface binding. After 45 minutes on ice, cells were washed twice, fixed for 5 minutes in 2% paraformaldehyde, and resuspended in PBS/0.5% BSA, analyzed by FACS and the FL2 geometric mean was used to quantitate inhibition of CXCR4 binding by 12G5-PE antibody.

Transient transfection. 293T cells were transiently transfected with the CXCR4 expression vector or control vector by Lipofectamine (Invitrogen, Carlsbad, Calif.), then treated with DV3-TAT-RxL or TATRxL peptide at 18 hours and the number of viable cells was counted 24 hr later. CXCR4 expression was quantified by 12G5-PE antibody treatment and FACS.

Statistical analysis. Student's t-test was used to determine statistical significance (p<0.05).

DV3 Enhances the Affinity of TAT Peptides for CXCR4 Expressing Cells. To determine if the addition of the DV3 ligand enhances the affinity of TAT peptides for CXCR4 expressing cancer cells, CXCR4 binding assays were carried out. Human Namalwa Burkitt's lymphoma cells overexpressing the CXCR4 receptor were treated with various peptides to block the CXCR4 receptor, then incubated with phycoerythrin (PE) conjugated anti-CXCR4 antibody and analyzed by flow cytometry.

Chemokines use two contact domains to bind their receptors, the first is represented by the DV3 peptide ligand (LGASWHRPDK—SEQ ID NO: 17) and the second is a basic patch mimicked by the TAT basic domain that facilitates the initial interaction with negatively charged chemokine receptors. Control DV3-only peptide displayed an IC₅₀ of ˜1 μM (FIG. 1B), a value that is within 2-fold of the published value. Consistent with chemokine two domain binding to CXCR4, the TAT basic peptide displayed a similar affinity as DV3 for CXCR4. However, linkage of DV3 and TAT basic domains resulted in a synergistic ˜100-fold increased affinity (IC₅₀<0.01 μM) for the CXCR4 receptor (FIG. 1B). Importantly, addition of the p53C′ cargo domain to the DV3-TAT peptide did not alter the affinity (IC₅₀<0.01 μM) for the CXCR4 receptor.

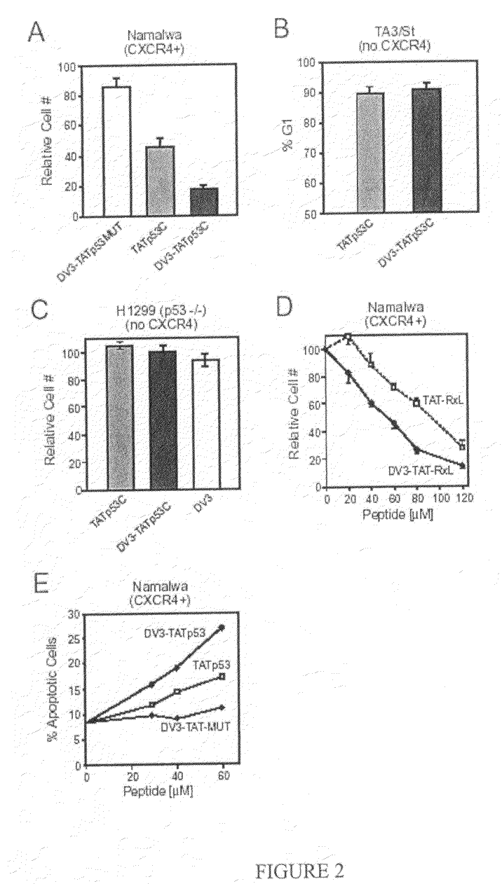

DV3-TATp53C′ and DV3-TAT-RxL Peptides have Enhanced Cell Killing in CXCR4-Expressing Tumor Cells. The ability of DV3-TATp53C′, DV3-TATp53MUT and parental TATp53C′ peptides to induce apoptosis in Namalwa lymphoma cells that overexpress the CXCR4 receptor were compared. TATp53C′ peptide treatment of Namalwa cells induced a dose-dependent decrease in cell number and concomitant increase in apoptotic cells (FIG. 2A,E). However, treatment with targeted DV3-TATp53C′ peptide resulted in an enhanced cell killing. This was particularly apparent at 40 μM where DV3-TATp53C′ peptide reduced cell number by >80%, whereas TATp53C′ peptide only reduced the cell number by 55% (FIG. 2A). In contrast, the functionally inactive, but transducible DV3-TATp53Mut peptide demonstrated background levels of activity on Namalwa cells (FIG. 2A). TA3/St mammary adenocarcinoma cells have undetectable CXCR4 surface expression and treatment of TA3/St cells with TATp53 C′ peptide induced a G1 arrest (FIG. 2B). Consistent with the absence of CXCR4 receptors, treatment of TA3/St cells with the targeted DV3-TATp53C′ peptide induced a G1 arrest that was indistinguishable from treatment with parental TATp53C′ peptide (FIG. 2B). In addition, targeted DV3-TATp53C′, parental TATp53C′ and control DV3 peptide had little to no effect on control, p53-deficient human H1299 lung adenocarcinoma cells (FIG. 2C).

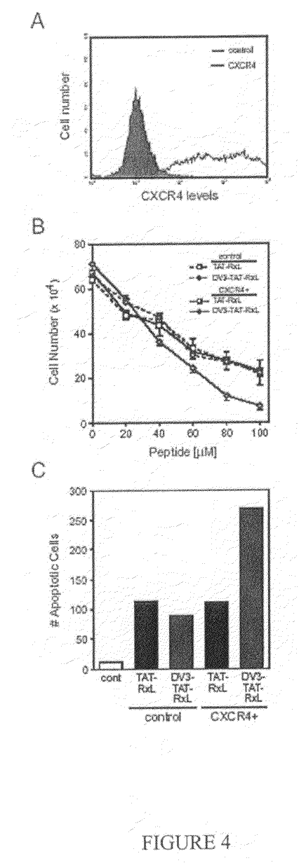

To test if the DV3 domain could enhance the activity of another proven anticancer peptide, a TAT-fusion peptide containing a domain that antagonizes Cdk2 activity was synthesized and termed TAT-RxL. Treatment of CXCR4 expressing Namalwa lymphoma cells with parental TAT-RxL reduced viable cell number in a dose-dependent fashion (FIG. 2D). However, treatment of Namalwa cells with the CXCR4-targeted, DV3-TAT-RxL peptide resulted in a significant increase in peptide potency at all concentrations tested. In contrast, TAT-RxL and DV3-TAT-RxL peptide treatment of non-CXCR4 expressing 293T cells (see FIG. 4B) and non-CXCR4 expressing TA3/St cells showed no differences between the two peptides. Taken together, these observations are consistent with the hypothesis that the DV3 domain enhances peptide delivery to CXCR4 overexpressing tumor cells.

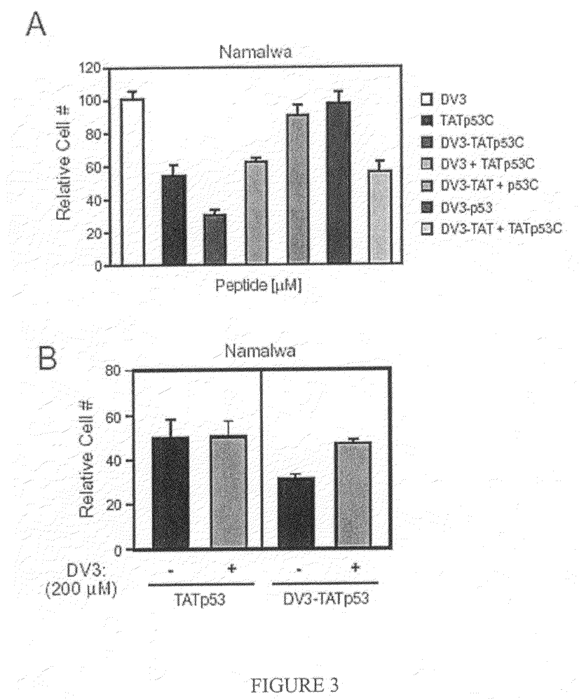

DV3 Domain Enhanced Killing of CXCR4 Expressing Cells Requires Covalent Linkage to TATp53C′ Peptide. To rule out the possibility that the enhanced potency of DV3-TATp53C′ was a consequence of CXCR4 blockade, a variety of control peptides were synthesized (FIG. 1A) and their ability to alter Namalwa lymphoma cell viability tested. Consistent With the observations above, targeted DV3-TATp53C′ peptide treatment of CXCR4 expressing Namalwa cells decreased viability to a significantly greater extent than treatment with parental TAT-p53C′ peptide (FIG. 3A). In contrast, treatment with control DV3 only peptide, DV3-p53 peptide or DV3-TAT peptide had minimal effects on cell number (FIG. 3A). Because the affinities of DV3-TAT and DV3-TATp53C′ for CXCR4 are nearly identical (FIG. 1B), these results suggested that the increased DV3-TATp53C′ activity cannot be explained purely by CXCR4 binding and antagonism.

Next assayed was whether enhanced DV3-TATp53C′ peptide activity could be reconstituted by adding its constituent domains to Namalwa cells in trans. Treatment of Namalwa cells with DV3 and TATp53C′ peptides in trans led to a similar reduction in cell viability as treatment with TATp53C′ peptide alone (FIG. 3A). Furthermore, coadministration of control DV3-TAT plus p53C′ peptides (FIG. 3A) or control DV3-p53C′ plus TAT peptide in trans also caused minimal to no cell death, and failed to reconstitute DV3-TATp53C′ in cis peptide activity. Finally, simultaneous treatment of lymphoma cells with DV3-TAT and TATp53C′ peptide reduced the cell number to the same extent as treatment with parental TATp53C′ peptide alone (FIG. 3A). Thus, regardless of the configuration, none of the DV3, TAT or p53C′ constituent domains, either alone or in trans, were as effective at killing CXCR4 expressing lymphoma cells as cis linked DV3-TATp53C peptide.

Enhanced DV3-TAT-RxL Peptide Killing of Tumor Cells Requires CXCR4. If the increased potency of DV3-TATp53C′ peptide was a direct result of the interaction between DV3-TATp53C′ peptide and the CXCR4 receptor, then elimination of this peptide/receptor interaction should reduce DV3-TATp53C′ peptide potency. To test this prediction, CXCR4 expressing Namalwa cells were incubated with DV3-TATp53C′ in the presence or absence of excess competing DV3 peptide. Addition of 200M excess DV3 peptide alone had no effect on Namalwa cell viability. However, co-administration of 200M DV3 peptide with 30M DV3-TATp53C′ peptide reduced the potency of DV3-TATp53C′ peptide to levels similar to that of parental TATp53C′ peptide (FIG. 3B). In contrast, excess DV3 only peptide had no effect on parental TATp53C′ peptide killing. These results suggested that DV3-TATp53C′ peptide interaction with CXCR4 is essential for increased potency; independent of disrupting CXCR4 signaling. CXCR4-targeted and parental non-targeted peptides have indistinguishable activities in non-CXCR4 expressing cells. Therefore, to directly test the requirement for CXCR4 overexpression for DV3-TAT domain enhancement, CXCR4 was ectopically expressed in non-CXCR4 expressing human 293T cells and assayed for altered peptide efficacies (FIG. 4A). Treatment of non-CXCR4 expressing 293T cells with parental TAT-RxL or targeted DV3-TAT-RxL peptides showed a near identical dose-dependent decrease in cell viability and induction of apoptosis (FIG. 4B,C). However, treatment of CXCR4 transfected 293T cells with the targeted DV3-TAT-RxL peptide resulted in an enhanced cell killing activity compared to treatment with parental TAT-RxL peptide at all concentrations tested (FIG. 4B). Taken together, these observations demonstrate that the cargo-independent, enhanced DV3-TATp53C′ and DV3-TAT-RxL activity derives from the increased targeting of the peptide via the DV3 domain to CXCR4 overexpressing cancer cells.

A number of embodiments have been described. Nevertheless, it will be understood that various modifications may be made without departing from the spirit and scope of the description. Accordingly, other embodiments are within the scope of the following claims.

What is claimed is:

1. A fusion polypeptide comprising:
a) a protein transduction domain (PTD), the transduction domain comprising a membrane transport function;
b) a ligand domain comprising a ligand specific for an extracellular polypeptide on a cell of interest; and
c) a heterologous domain,
wherein the PTD is operably linked to the ligand domain and the heterologous domain.

2. The fusion polypeptide of claim 1, wherein the protein transduction domain is selected from the group consisting of a polypeptide comprising a herpesviral VP22 domain; a polypeptide comprising a human immunodeficiency virus (HIV) TAT domain; a polypeptide comprising a homeodomain of an Antennapedia protein (Antp HD) domain; an N-terminal cationic prion protein domain; and functional fragments thereof.

3. The fusion polypeptide of claim 1, wherein the protein transduction domain comprises a sequence selected from the group consisting of SEQ ID NO:7 from amino acid 47-57; B1-X₁-X₂-X₃-B₂-X₄-X₅-B₃, wherein B₁, B₂, and B₃ are each independently a basic amino acid, the same or different and X₁, X₂, X₃, X₄ and X₅ are each independently an alpha-helix enhancing amino acid the same or different (SEQ ID NO: 1); B₁-X₁-X₂-B₂-B₃-X₃-X₄-B₄, wherein B₁, B₂, B₃, and B₄ are each independently a basic amino acid, the same or different and X₁, X₂, X₃, and X₄ are each independently an alpha-helix enhancing amino acid the same or different (SEQ ID NO:2); X-X-R-X-(P/X)-(B/X)-B-(P/X)-X-B-(B/X), wherein X is any alpha helical promoting residue such as alanine; P/X is either proline or X as previously defined, B is a basic amino acid residue and B/X is either B or X as defined above (SEQ ID NO:4); a sequence of about 7 to 10 amino acids and containing KX₁RX₂X₁, wherein X₁ is R or K and X₂ is any amino acid (SEQ ID NO:5); RKKRRQRRR (SEQ ID NO:6); and KKRPKPG (SEQ ID NO:3).

4. The fusion polypeptide of claim 1, wherein the heterologous domain comprises a diagnostic and/or therapeutic agent.

5. The fusion polypeptide of claim 4, wherein the therapeutic agent is a thrombolytic agent or an anticellular agent.

6. The fusion polypeptide of claim 5, wherein the thrombolytic agent comprises streptokinase or urokinase.

7. The fusion polypeptide of claim 4, wherein the therapeutic agent is an anticellular agent.

8. The fusion polypeptide of claim 7, wherein the anticellular agent is selected from the group consisting of a chemotherapeutic agent and a mammalian cell cytotoxin.

9. The fusion polypeptide of claim 8, wherein the chemotherapeutic agent is selected from the group consisting a steroid, an antimetabolite, an anthracycline, an vinca alkaloid, an antibiotic, an alkylating agent, an epipodophyllotoxin, neocarzinostatin (NCS), adriamycin and dideoxycytidine.

10. The fusion polypeptide of claim 8, wherein the mammalian cell cytotoxin is selected from the group consisting of interferon-α (IFN-α), interferon-βγ (IFN-βγ), interleukin-12 (IL-12) and tumor necrosis factor-α (TNF-α).

11. The fusion polypeptide of claim 7, wherein the anticellular agent is selected from the group consisting of plant-, fungus- and bacteria-derived toxins.

12. The fusion polypeptide of claim 11, wherein the toxin is selected from the group consisting of a ribosome inactivating protein, gelonin, a-sarcin, aspergillin, restrictocin, ribonucleases, diphtheria toxin, Pseudomonas exotoxin, bacterial endotoxins, the lipid A moiety of a bacterial endotoxin, ricin A chain, deglycosylated ricin A chain and recombinant ricin A chain.

13. The fusion polypeptide of claim 7, wherein the therapeutic agent comprises a radioactive moiety comprising a radioisotope.

14. The fusion polypeptide of claim 4, wherein the therapeutic agent is an anti-cancer agent.

15. The fusion polypeptide of claim 14, wherein the anti-cancer agent inhibits cell proliferation.

16. The fusion polypeptide of claim 14, wherein the anti-cancer agent is a suicide gene or a tumor suppressor protein.

17. The fusion polypeptide of claim 16, wherein the suicide gene is thymidine kinase.

18. The fusion polypeptide of claim 16, wherein the tumor suppressor protein is p53.

19. The fusion polypeptide of claim 4, wherein the diagnostic agent is selected from the group consisting of a fluorgenic agent, a paramagnetic agent and a radioactive agent.

20. The fusion polypeptide of claim 19, wherein the paramagnetic agent comprises an ion selected from the group consisting of chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (II) ions.

21. The fusion polypeptide of claim 19, wherein the radioactive agent comprises an ion selected from the group consisting of iodine¹²³, technicium^{99m}, indium¹¹¹, rhenium¹⁸⁸, rhenium¹⁸⁶, copper⁶⁷, iodine¹³¹, yttrium⁹⁰, iodine¹²⁵, astatine²¹¹, gallium⁶⁷, iridium¹⁹², cobalt⁶⁰, radium²²⁶, gold¹⁹⁸, cesium¹³⁷ and phosphorus³² ions.

22. The fusion polypeptide of claim 19, wherein the fluorogenic agents is selected from the group consisting of gadolinium and renographin.

23. The fusion polypeptide of claim 1, wherein the ligand binds to a cell surface protein selected from the group consisting of melanocortin receptor (MC1), αv integrins, αvβ3 integrin, αvβ6 integrin, α4 integrins, α5 integrins, α6 integrins, α9 integrins, CD13, melanoma proteoglycan, membrane dipeptidase (MDP), TAG72 antigen, an antigen binding site of a surface immunoglobulin receptor of B-cell lymphomas, type I interleukin I (IL-1) receptor, human immunodeficiency virus type 1 (HIV-1) envelope glycoprotein (gp120), atrial natriuretic peptide (ANP) receptor, erythropoietin (EPO) receptor, thrombopoietin (TPO) receptor, carcino-embryonic antigen (CEA) receptor, EpCAM, CD40, prostate-specific membrane antigen (PSMA), endoglin (CD105), epidermal growth factor receptor (EGFR), HER2, CXCR4, LHRH receptor, and extracellular matrix components.

24. A pharmaceutical composition comprising the fusion polypeptide of claim 1.

25. A method of introducing a therapeutic and/or diagnostic agent in to a target cell, the method comprising contacting the cell with the fusion polypeptide of claim 1.

26. The method of claim 25, wherein the contacting is in vivo or in vitro.

27. The method of treating a cell proliferative disorder in a subject, comprising contacting the subject with a fusion polypeptide of claim 1, wherein the heterologous domain comprises an anticellular agent.

28. The method of claim 27, wherein the ligand domain comprises a ligand that binds to a cell surface marker expressed on a cell comprising a cell proliferative disorder.

29. The method of claim 28, wherein the ligand domain comprises DV3.

30. A method of identifying a cell comprising a phenotype of interest in a subject, the method comprising contacting the subject with a fusion polypeptide of claim 1, wherein the heterologous domain comprises a diagnostic agent.

31. An isolated polynucleotide encoding the fusion polypeptide of claim 1.

32. A vector comprising the polynucleotide of claim 31.

33. A host cell containing the vector of claim 32.

34. A host cell containing the polynucleotide of claim 31.
